(19) United States
(12) Patent Application Publication
(10) Pub. No.: US 2025/0281323 A1
(43) Pub. Date: Sep. 11, 2025

(54) METHODS OF USE OF PRECISION AND CONTROLLED COOLING DEVICES IN COMBINATION WITH OTHER TREATMENTS

(71) Applicant: RECENSMEDICAL, INC., Hwaseong-si, Gyeonggi-do (KR)

(72) Inventor: Gun-Ho KIM, Hwaseong-si, Gyeonggi-do (KR)

(21) Appl. No.: 18/851,006

(22) PCT Filed: Mar. 30, 2023
(86) PCT No.: PCT/IB2023/000172

§ 371 Date: Sep. 25, 2024

Related U.S. Application Data
(60) Provisional application No. 63/326,140, filed on Mar. 31, 2022.

(51) Int. Cl. A61F 7/00

(52) U.S. Cl. CPC A61F 7/00; A61F 2007/0052; A61F 2007/0063; A61F 2007/0087; A61F 2007/0095

(57) ABSTRACT

Proposed is a method of rapidly and precisely cooling a skin area of a subject in combination with a treatment of the skin area. The method may include providing a handheld cooling device. The device may include an applicator tip, and a controlled cryogen spray element configured to spray controlled cryogen. The method may also include contacting the tip on the skin area, spraying the controlled cryogen onto the skin area, and applying the treatment to the skin area.

### CROSS-REFERENCE

This application claims the benefit of U.S. Provisional Patent Application No. 63/326,140 filed on Mar. 31, 2022, which is incorporated by reference in its entirety.

### BACKGROUND

Controlled cooling devices offer advantages over convention means of providing analgesic, anesthetic, or surgical applications. There is a desire for providers to provide comfortable experiences and superior outcomes to their patients for a variety of treatments and applications, which may include reduction in pain, bruising, swelling, inflammation, or excessive bleeding. In addition, there is a desire for providers to understand the parameters to achieve these outcomes or experiences while minimizing their workflow disruption and mitigating adverse reactions to the skin related to uncontrolled cooling (e.g., post-inflammatory hyperpigmentation). Accordingly, clinical protocols for appropriately use of controlled cooling devices alongside different treatments in these application are needed.

### SUMMARY

In one aspect, described herein, are methods of rapidly and precisely cooling a skin area of a subject in combination with a treatment of the skin area. In some aspects, the method comprises a providing a handheld cooling device. In some aspects, the device comprises an applicator tip. In other aspects, the device comprises a controlled cryogen spray element, wherein the controlled cryogen spray element is configured to spray controlled cryogen. In some aspects, the method comprises contacting the tip on the surface area. In some aspects, the method further comprises spraying controlled cryogen onto the skin area. In some aspects, the method comprises applying the treatment to the skin area.

In some aspects, the treatment comprises an injection, a dermal filler treatment, a neurotoxin treatment, a PRP injection, a lidocaine injection, a skin booster injection, a biopsy, a piercing treatment, a chemical peel treatment, a microneedling treatment, an RF microneedling treatment, a laser treatment, an energy based device treatment, a blood draw, a nerve block, an incision, or a surgery.

In some aspects, the device further comprises (a) a user interface configured to receive a user input of a target temperature; (b) a real-time temperature sensor configured to detect the real-time temperature of the skin area; and an alert element configured to provide a target temperature alert when the detected real-time temperature of the skin area has reached the target temperature. In some aspects, there may be a plurality of real-time temperature sensors in the device. In some aspects. the target temperature alert comprises a visual indicator, an auditory indicator, a physical vibration, a single beep, a plurality of beeps, a visual light, or any combination thereof. In other aspects, the method further comprises receiving the target temperature, detecting the real-time temperature of the skin area, and providing the target temperature alert. In some aspects, the method further comprises applying the treatment after providing the target temperature alert. −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., or 10° C.

In some aspects, the user element is further configured to receive a user input of a duration of time and the alert element is further configured to provide a duration alert when the real-time temperature sensor detects that the skin area has maintained the target temperature for the duration of time. In some aspects, the method further comprises providing the duration alert. In some aspects, the method further comprises applying the treatment after providing the duration alert. In some aspects, the duration of time comprises 1 second, 2 seconds, 3 seconds, 4seconds, 5 seconds, 6 seconds, 7 seconds, 8 seconds, 9 seconds, or 10 seconds. In some aspects, the device is further configured to receive a preset temperature or time that is displayed to the user in a quick fashion (e.g.,, “Quick” or “EZ” on an LCD display to mean 2 seconds).

In some aspects, the method further comprises stopping the spray of controlled cryogen onto the skin area before or during the treatment. In some aspects, the stop is made after the treatment. In some aspects, the tip comprises an open portion. In some aspects, the method comprises applying the treatment to the skin through the opened portion. In some aspects, the device comprises a cryogen receive element configured to receive cryogen. In some aspects, the method comprises orienting the device so the received cryogen flows toward a direction of gravity.

In some aspects, the device comprises a treatment alert configured to notify the user when to eperform the treatment. In another aspect, the skin area comprises a lip, a facial area, a hand, a foot, an arm, a nose, a knee, an elbow, a leg, an area on a body, a neck, or a scalp. In some aspects, the method further comprises replacing the tip that contacted to or around the skin area with a new applicator tip after applying the treatment to the skin area. In some aspects, the subject is a human, a mouse, a pig, a dog, a cat, or a mammal. In some aspects, the method comprises contacting the tip to a different skin area while the controlled cryogen is spraying, spraying controlled cryogen onto the different skin area, and applying the treatment to the different skin area.

In some aspects, the device comprises a cartridge status alert, which is configured to alert the user when the cryogen is almost fully spent. In some aspects, the device comprises an orientation status alert, which is configured to alert the user when the user is not properly orienting the device in the proper orientation (e.g., if the device is not being held so that the contents of a cartridge is pointed downward to allow the cryogen to flow downward with the flow of gravity into the device and out of the controlled cryogen spray element to the skin target area). In some aspects, the device cartridge status alert and the orientation status alert come from the same element within the device. In some aspects, the device comprises a timer, which is configured to notify the user how much total elapsed time of cooling has been used so the user can understand when the cartridge is empty or close to being empty and replacement of a cartridge is appropriate prior to starting a treatment with a new patient.

In one aspect, described herein are methods of reducing discomfort of a subject during a treatment to a skin area of the subject. In some aspects, the method comprises providing a handheld cooling device. In some aspects, the cooling device comprises an inclined applicator tip. In some aspects, the inclined applicator tip comprises an opened portion. In some aspects the inclined applicator tip comprises an elongated body portion coupled to the opened portion. In some aspects, the inclined applicator tip comprises a mounting portion adapted to be operably coupled to the handheld cooling device.

In some aspects, the method comprises contacting the applicator tip to or around the skin area. In some aspects, the method comprises cooling the skin area to a cooling temperature. In some aspects, the method comprises applying the treatment to the skin area by providing an access of the treatment through the opened portion. In some aspects, the treatment is an injection, a lip filler treatment, a piercing treatment, a laser treatment, an energy based device, a blood draw, or a surgery. In some aspects, the skin area comprises irregular surface. In some aspects the skin area is a lip, eyelid, ear, nose, face, or neck. In some aspects, the contacting comprises contacting a tip of the opened portion to or around the skin area. In some aspects, the circumference of the opened portion partially surrounds the skin area. In some aspects, the circumference of the opened portion is within 5 mm, 10 mm, 15 mm, 20 mm, or 25 mm from the skin area of the treatment.

In some aspects, the cooling temperature is between about 0 degree to 10 degree Celsius. In some aspects, the cooling temperature is between 3 degree to 8 degree Celsius. In some aspects, the temperature of the skin area is maintained at the cooling temperature for at least 2sec, 5 sec, 10 sec, 15 sec, or 20 sec.

In another aspect, the method further comprises selecting an orientation of the opened portion with respect to a cooling axis of the handheld cooling device. In some aspects, the orientation of the opened portion forms an angle of inclination (θ) with respect to the cooling axis. In some aspects, the angle of inclination and the selected orientation are designed to minimize a reduction of cryogen flow into the handheld cooling device caused by gravity.

In another aspect, described herein are methods of reducing discomfort of a subject after a treatment to a skin area of the subject. In some aspects, the method comprises providing a handheld cooling device. In some aspects, the cooling device comprises an inclined applicator tip. In some aspects, the inclined applicator tip comprises an opened portion, an elongated body portion coupled to the opened portion, and a mounting portion adapted to be operably coupled to the handheld cooling device. In some aspects, the method comprises contacting the applicator tip to or around the skin area. In some aspects, the method comprises applying the treatment to the skin area by providing an access of the treatment through the opened portion. In some aspects, the method comprises cooling the skin area to a cooling temperature.

In some aspects, the treatment is an injection, a laser treatment, an energy based device, or a surgery. In some aspects, the discomfort comprises swelling of the skin area or a pain in the skin area. In some aspects, the skin area comprises irregular surface. In some aspects, the skin area is eyelid, ear, nose, face, or neck. In some aspects, the contacting comprises contacting a tip of the opened portion to or around the skin area. In some aspects, the circumference of the opened portion partially surrounds the skin area. In some aspects, the circumference of the opened portion is within 5 mm, 10 mm, 15 mm, 20 mm, or 25 mm from the skin area of the treatment. In some aspects, the cooling temperature is between about 0 degree to 10 degree Celsius.

In some aspects, the cooling temperature is between 3 degree to 8 degree Celsius. In some aspects, the temperature of the skin area is maintained at the cooling temperature for at least 1 sec, 2 sec, 5 sec, 10 sec, 15 sec, or 20 sec. In some aspects, cooling the skin area to a cooling temperature is performed within 1 sec, 2 sec, 3 sec, 4 sec, 5 sec, 10 sec, or 15 sec after the treatment. In some aspects, cooling the skin area to a cooling temperature is performed for a duration of 1 sec, 2 sec, 3 sec, 4 sec, 5 sec, 10 sec, or 15 sec. In some aspects, cooling the skin area to a cooling temperature is performed plurality of times with an intermittent break period. In some aspects, the intermittent break period is at least 1 sec, 2 sec, 5 sec, 10 sec, 15 sec, or 20 sec. In some aspects, cooling the skin area to a cooling temperature is performed for the same duration for the plurality of times. In some aspects, cooling the skin area to a cooling temperature is performed for the different durations.

Additional aspects and advantages of the present disclosure will become readily apparent to those skilled in this art from the following detailed description, wherein only illustrative aspects of the present disclosure are shown and described. As will be realized, the present disclosure is capable of other and different aspects, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative aspects, in which the principles of the invention are utilized, and the accompanying drawings (also “Figure” and “FIG.” herein), of which:

FIG. 1A illustrates correct alignment of the cooling jet of a cooling device with the infrared sensor beam of a cooling device, when a target surface is flat. FIG. 1B illustrates slight misalignment of the cooling jet and sensor beam when the target surface is not flat.

FIG. 2 illustrates example alignment of a cooling device and an injection needle, wherein the target surface is an arm of a subject and cooling and injection are performed simultaneously.

### DETAILED DESCRIPTION

While various aspects of the invention have been shown and described herein, it will be obvious to those skilled in the art that such aspects are provided by way of example only. Numerous variations, changes, and substitutions can occur to those skilled in the art without departing from the invention. It should be understood that various alternatives to the aspects of the invention described herein can be employed.

### Terms and Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”.

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some aspects of any of the compositions and methods provided herein, “comprising” can be replaced with “consisting essentially of” or “consisting of.” The phrase “consisting essentially of” is used herein to require the specified feature(s) as well as those which do not materially affect the character or function of the claimed disclosure. As used herein, the term “consisting” is used to indicate the presence of the recited feature alone. As used herein, the term “and/or” includes any and all combinations of one or more of the associated listed items.

In the drawings, the thicknesses of layers and regions are exaggerated for clarity, and elements or layers are referred to as “on” or “on” the other elements or layers includes not only cases where they are directly above the other components or layers, but also cases where there are other layers or other components interposed in the middle. The same reference numerals throughout these specifications refer to the same elements unless indicated otherwise. In addition, components that have the same functions within the scope of the same idea shown in the drawings of each embodiment are described using the same reference numerals, and redundant descriptions thereof are omitted.

The numbers (e.g., first, second, etc.) used in the description of the present disclosure are merely identification symbols for distinguishing one component from other components.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Thus, the term “thermally coupled” refers any relationship between two materials or objects where the heat is transferred by direct or indirect contact (e.g., via vapor, air, liquid, mechanical forces, etc.).

In some aspects, the term ‘target’ refers to an object that is cooled by using a cooling system. For example, in some aspects, the target is a target intended to be put into an anesthetic state or a painless state in order to receive a medical procedure. Specifically, the target comprises a part of a body including nerves, such as eyes, skin, or gum with disease. In another example, the target comprises a target for skin cosmetic treatment using cooling. Specifically, the target can comprise a part of a body including a point that can be removed by cooling a local area, warts, callus, etc., or a part of a body requiring local anesthesia during an energy based device treatment, laser treatment, or other treatments such as hair removal, peeling, and Botox treatment. In some aspects, the target can include cells or tissues for scientific experiments (e.g., cell culture, tissue culture, cultured cells, cultured tissue). Alternatively, and/or additionally, the target can include any containers (e.g., glass vial, glass substrate, petri dish, etc.) containing cells or tissues.

As used herein, the term ‘cooling’ refers to reducing the temperature of a target to be cooled. In some aspects, the cooling is accomplished by applying cooling energy to the target to be cooled and absorbing the heat energy of the target to be cooled. In some aspects, the cooling energy can refer to the escaping of heat by cooling and can be understood as a concept for expressing the decrease of thermal energy. For example, cooling can be conducted by applying cooling energy through a cooling medium, which in some aspects can further cool a contact tip, or by applying cooling energy to the tip, and ‘contacting’ the cooling medium and/or a cooled tip to a target to be cooled so that the cooling energy can be transmitted or applied to the target.

As used herein, the term ‘controlled cryogen’ refers to cryogen that has its thermodynamic state altered and/or regulated by having applied to the cryogen. In some aspects, the applied heat originates from within the cooling system. In other aspects, the applied heat may originate from a source outside the cooling system.

In another example, cooling can be conducted by ‘injecting’ or jetting a coolant, air, gas, or a liquid cryogen to apply cooling energy to the target to be cooled. Injecting or jetting can include producing a controlled aerosol spray of liquid cryogen directly or indirectly on to a target surface to be cooled. In other words, it should be understood as a comprehensive concept including various methods of applying cooling energy to an object to be cooled. Hereinafter, for convenience of description, cooling a target through a non-contact cooling device which operates by applying a cryogen spray to the target is described as a main embodiment, but the technical idea of the present specifications is not limited thereto.

As used herein, “cooling axis” generally refers to the line along which a therapeutic cooling device operates. For example, in a cryogen-based cooling device, the cooling axis is the direction of the cryogen spray. Examples of cryogen-based cooling devices include devices which apply a spray or aerosol of a liquified cryogen-gas such as but not limited to liquid He, Ar, N₂, O₂, CO₂, H₂, methane, ethane, propane, or combinations or mixtures thereof to reduce the temperature of surfaces that the spray or aerosol comes into contact with.

As used herein, “angle of inclination (θ)” is measured as the angle between the longitudinal cooling axis of a therapeutic cooling device and the surface of a target tissue when the cooling device is operably coupled to an inclined applicator tip. An inclined applicator tip can set the angle of inclination by placing the contact surface of the applicator tip at the angle of inclination with respect to the longitudinal cooling axis of the therapeutic device.

As used herein, “non-contact” refers to a cooling device which does not rely on direct induction of energy between a heat exchanger and the target site. Examples of non-contact cooling devices include cryogen spray-based cooling devices. Importantly, non-contact does not indicate that no part of the device is in contact with the subject or target site. Non-contact cooling devices can include guide tips, such as those described herein, which have a contact surface and can be used to fix the distance between a non-contact cooling element, such as a cryogen spray nozzle, and a target site. In some aspects, the tips may be configured to adjust the distance between a non-contact cooling element, such as a cryogen spray nozzle, and a target site.

As used herein, “simultaneous” refers to events that occur at substantially the same time, wherein two events that within a second of each other are simultaneous.

As used herein, “near-simultaneous” refers to an event occurring at a time that is no more than about 60 seconds after an application of a therapeutic cooling device as measured from the completion of a full therapeutic cooling cycle. For example, an event occurring about 1 second to about 60 seconds after the completion of the cooling cycle would be “near-simultaneous”.

As used herein, ranges and amounts can be expressed as “about” a particular value or range. About also includes the exact amount. Hence “about 5° C.” means “about 5° C.” and also “5° C.” Generally, the term “about” includes an amount that is expected to be within experimental error. For example, if the expected measurement error is +/−10%, “about 5° C.” means “5° C.+/−0.5° C.”

### Exemplary Cooling Devices and Applicator Tips

In one aspect, described herein are devices for use with a cooling device.

In one aspect, described herein are inclined applicator tips adapted to be operably coupled to a cooling device. In some instances, the cooling device is a handheld cooling device. In some instances, the cooling device is a therapeutic cooling device. In some instances, the handheld cooling device is a therapeutic cooling device. In some instances, the cooling device is a cryogenic cooling device. In some aspects, the inclined applicator tips comprise an opened portion wherein at least a portion of an edge of the opened portion comprises a c-shape opening. In some aspects, the inclined applicator tips comprise an elongated body portion coupled to the opened portion, wherein the longest dimension of the elongated body portion forms an angle of inclination (θ) with respect to the plane which is parallel to the c-shape opening. In some aspects, the elongated body portion is substantially parallel to the cooling axis of the handheld cooling device when operably coupled, and a mounting portion adapted to be operably coupled to the therapeutic cooling device. In some aspects, the mounting portion and the opened portion are coupled to the elongated body portion at opposite ends of the longest dimension of the elongated body portion.

In some aspects, the inclined applicator tip comprises an opened portion which comprises a circular, an elliptical, or a polygonal shape. In some aspects, the inclined applicator tip comprises an opened portion which comprises a circular shape wherein the portion of the circular shape which forms the opening comprises a 90° arc. In some aspects, the opened portion comprises a circular shape and the portion of the circular shape which forms the opening comprises an 180° arc. In some aspects, the opened portion comprises a circular shape and the portion of the circular shape which forms the opening comprises a 270° arc.

In some aspects, the angle of inclination (θ) is about 60°. In some aspects, the angle of inclination (θ) is about 65°. In some aspects, the angle of inclination (θ) is about 70°. In some aspects, the angle of inclination (θ) is about 75°. In some aspects, wherein the angle of inclination (θ) is about 80°. In some aspects, the angle of inclination (θ) is about 85°. In some aspects, the angle of inclination (θ) is about 90°. In some embodiments, the opened portion comprises an inner radius of at least 5 mm, 10 mm, 15 mm, 20 mm, 25 mm, 30 mm, 35 mm, 40mm, 45 mm, or 50 mm.

In some aspects, the longest dimension of the elongated body portion is no more than about 150 mm, about 200 mm, about 250 mm, about 300 mm. In some aspects, the longest dimension of the elongated body portion is at least about 15 mm, at least about 20 mm, at least about 25 mm, at least about 30 mm, at least about 35 mm, at least about 40 mm, at least about 45 mm, at least about 50 mm. In some aspects, the handheld cooling device is a cryogenic cooling device.

In some aspects, the opened portion comprises a circular shape and the portion of the circular shape which forms the opening with a 90° arc. In some aspects, the opened portion comprises a circular shape and the portion of the circular shape which forms the opening with a 180° arc. In some aspects, the angle of inclination (θ) is about 60° and the opened portion comprises an inner radius of at least 5 mm.

In some aspects, the mounting portion comprises a magnetic coupling element adapted to be operably coupled to the therapeutic cooling device. In some aspects, the magnetic coupling element is one or more permanent magnets. In some aspects, the magnetic coupling elements are made of a ferromagnetic material, and the therapeutic cooling device comprises one or more magnets configured to attract the ferromagnetic material when the inclined applicator tip is coupled to the device.

### Use of a Controlled Cooling Device

Also described herein are methods for use of devices described herein to provide cooling to a subject. In some instances, the cooling device is the cooling device described herein. In some instances, the cooling device can be any cooling device that can control the temperature of the target surface precisely (e.g., within about ±5° C., within about ±4° C., within about ±3° C., within about ±2° C., within about ±1° C., within about ±0.5° C., within about ±0.1° C.) after cooling medium is contacted to the target surface. In some instances, the cooling device can be any cooling device that can control the temperature of the target surface precisely (e.g., within about ±5° C., within about ±4° C., within about ±3° C., within about ±2° C., within about ±1° C., within about ±0.5° C., within about ±0.1° C.) during at least 70%, at least 80%, at least 90%, at least 95% of the duration time of the treatment.

In some aspect described herein are methods of cooling a target surface using a handheld cooling device. In some aspects, the handheld device comprises, or coupled to, an inclined applicator tip that is adapted to be operably coupled to the handheld cooling device. In some instances, the inclined applicator tip comprises an opened portion, an elongated body portion coupled to the opened portion, and a mounting portion adapted to be operably coupled to the handheld cooling device.

In some aspects, an orientation of the opened portion can be selected with respect to a cooling axis of the handheld cooling device, such that the orientation of the opened portion forms an angle of inclination (θ) with respect to the cooling axis. Once the orientation is selected, the target surface can be cooled by delivering the cooling medium from the handheld cooling device to the target surface. In some instances, the cooling medium can be delivered to the target surface substantially along the cooling axis. In some instances, the cooling medium can be delivered to the target surface by spreading along the cooling axis, for example by diffusion of a cooling jet.

In some aspects, selection of the orientation of the opened portion is performed by mounting the applicator tip to the handheld cooling device in a position selected from three discreet rotational orientations. In some aspects, the selection of the orientation of the opened portion is performed by selecting an orientation from a continuum of rotational orientations using a rotatable mount of the handheld cooling device. For example, a device comprising an inclined applicator tip with a fixed angle of inclination and a rotatable mount can accommodate target tissues which are tilted to any orientation, such as tilted up or tilted down with opposite magnitudes. In this example, the orientation of the device can be maintained with respect to gravity by rotating the tip 180°.

In some aspects, the cooling medium is a cryogen. In some instances, a cryogen is any appropriate substance that can cool down the target surface to about −5° C., about −4° C., about −3° C., about −2° C., about −1° C., about 0° C., about 1° C., about 2° C., about 3° C., about 4° C., or about 5° C. In some instances, the cooling device can cool down the target surface to preferably close to 0° C., but not lower than 0° C. (e.g., about 0° C., about 1° C., about 2° C., about 3° C., about 4° C., or about 5° C.) to avoid post-inflammatory hyperpigmentation of the target surface (e.g., portion of the skin). In some aspects, the cooling medium is controlled cryogen.

In some aspects, a controlled cooling device can be configured to precisely control a temperature of a target surface. In some aspects, a controlled cooling device can be configured to maintain a temperature of a target surface for a set period of time.

In some aspects, the temperature is controlled to a target temperature of zero ° C. in order to minimize post-inflammatory hyperpigmentation (which can be caused if the target surface is made too cold for too long). In some aspects, a combination of target temperature and cooling duration (e.g. the time that a target temperature is maintained) is chosen to minimize post-inflammatory hyperpigmentation. For example, cooler temperatures (less than zero) can be used with a shorter cooling duration (e.g. two seconds) or higher temperatures can be used with a longer duration (e.g. 3° C. for five seconds).

In some aspects, the temperature is controlled to about 0° C. to about 8° C. In some aspects, the temperature is controlled to about 0° C. to about 1° C., about 0° C. to about 2° C., about 0° C. to about 3° C. about 0° C. to about 4° C., about 0° C. to about 5° C., about 0° C. to about 6° C., about 0° C. to about 7° C., about 0° C. to about 8° C., about 1° C. to about 2° C., about 1° C. to about 3° C., about 1° C. to about 4° C., about 1° C. to about 5° C., about 1° C. to about 6° C., about 1° C. to about 7° C., about 1° C. to about 8° C., about 2° C. to about 3° C., about 2° C. to about 4° C., about 2° C. to about 5° C., about 2° C. to about 6° C., about 2° C. to about 7° C., about 2° C. to about 8° C., about 3° C. to about 4° C., about 3° C. to about 5° C., about 3° C. to about 6° C., about 3° C. to about 7° C., about 3° C. to about 8° C., about 4° C. to about 5° C., about 4° C. to about 6° C., about 4° C. to about 7° C., about 4° C. to about 8° C., about 5° C. to about 6° C., about 5° C. to about 7° C., about 5° C. to about 8° C., about 6° C. to about 7° C., about 6° C. to about 8° C., or about 7° C. to about 8° C. In some aspects, the temperature is controlled to about 0° C., about 1° C., about 2° C., about 3° C., about 4° C., about 5° C., about 6° C., about 7° C., or about 8° C. In some aspects, the temperature is controlled to at least about 0° C., about 1° C., about 2° C., about 3° C., about 4° C., about 5° C., about 6° C., or about 7° C. In some aspects, the temperature is controlled to at most about 1° C., about 2° C., about 3° C., about 4° C., about 5° C., about 6° C., about 7° C., or about 8° C.

In some aspects, the temperature is controlled to about −10° C. to about 1° C. In some aspects, the temperature is controlled to about −10° C. to about −9° C., about −10° C. to about −8° C., about −10° C. to about −7° C., about −10° C. to about −6° C., about −10° C. to about −5° C., about −10° C. to about −4° C., about −10° C. to about −3° C., about −10° C. to about −2° C., about −10° C. to about −1° C., about −10° C. to about 0° C., about −10° C. to about 1° C., about −9° C. to about −8° C., about −9° C. to about −7° C., about −9° C. to about −6° C., about −9° C. to about −5° C., about −9° C. to about −4° C., about −9° C. to about −3° C., about −9° C. to about −2° C., about −9° C. to about −1° C., about −9° C. to about 0° C., about −9° C. to about 1° C., about −8° C. to about −7° C., about −8° C. to about −6° C., about −8° C. to about −5° C., about −8° C. to about −4° C., about −8° C. to about −3° C., about −8° C. to about −2° C., about −8° C. to about −1° C., about −8° C. to about 0° C., about −8° C. to about 1° C., about −7° C. to about −6° C., about −7° C. to about −5° C., about −7° C. to about −4° C., about −7° C. to about −3° C., about −7° C. to about −2° C., about −7° C. to about −1° C., about −7° C. to about 0° C., about −7° C. to about 1° C., about −6° C. to about −5° C., about −6° C. to about 4° C., about −6° C. to about −3° C., about −6° C. to about −2° C., about −6° C. to about −1° C., about −6° C. to about 0° C., about −6° C. to about 1° C., about −5° C. to about −4° C., about −5° C. to about −3° C., about −5° C. to about −2° C., about −5° C. to about −1° C., about −5° C. to about 0° C., about −5° C. to about 1° C., about −4° C. to about −3° C., about −4° C. to about −2° C., about −4° C. to about −1° C., about −4° C. to about 0° C., about −4° C. to about 1° C., about −3° C. to about −2° C., about −3° C. to about −1° C., about −3° C. to about 0° C., about −3° C. to about 1° C., about −2° C. to about −1° C., about −2° C. to about 0° C., about −2° C. to about 1° C., about −1° C. to about 0° C., about −1° C. to about 1° C., or about 0° C. to about 1° C. In some aspects, the temperature is controlled to about −10° C., about −9° C., about −8° C., about −7° C., about −6° C., about −5° C., about −4° C., about −3° C., about −2° C., about −1° C., about 0° C., or about 1° C. In some aspects, the temperature is controlled to at least about −10° C., about −9° C., about −8° C., about −7° C., about −6° C., about −5° C., about −4° C., about −3° C., about −2° C., about −1° C., or about 0° C. In some aspects, the temperature is controlled to at most about −9° C., about −8° C., about −7° C., about −6° C., about −5° C., about −4° C., about −3° C., about −2° C., about −1° C., about 0° C., or about 1° C.

In some aspects, the temperature is controlled to about −80° C. to about −10° C.

In some aspects, the target cooling temperature is maintained for about 1 second to about 12 seconds. In some aspects, the target cooling temperature is maintained for about 1 second to about 2 seconds, about 1 second to about 3 seconds, about 1 second to about 4 seconds, about 1 second to about 5 seconds, about 1 second to about 6 seconds, about 1 second to about 7 seconds, about 1 second to about 8 seconds, about 1 second to about 9 seconds, about 1 second to about 10 seconds, about 1 second to about 11 seconds, about 1 second to about 12 seconds, about 2 seconds to about 3 seconds, about 2 seconds to about 4 seconds, about 2 seconds to about 5 seconds, about 2 seconds to about 6 seconds, about 2 seconds to about 7 seconds, about 2 seconds to about 8 seconds, about 2 seconds to about 9 seconds, about 2 seconds to about 10 seconds, about 2 seconds to about 11 seconds, about 2 seconds to about 12 seconds, about 3 seconds to about 4 seconds, about 3 seconds to about 5 seconds, about 3 seconds to about 6 seconds, about 3 seconds to about 7 seconds, about 3 seconds to about 8 seconds, about 3 seconds to about 9 seconds, about 3 seconds to about 10 seconds, about 3 seconds to about 11 seconds, about 3 seconds to about 12 seconds, about 4 seconds to about 5 seconds, about 4 seconds to about 6 seconds, about 4 seconds to about 7 seconds, about 4 seconds to about 8 seconds, about 4 seconds to about 9 seconds, about 4 seconds to about 10 seconds, about 4 seconds to about 11 seconds, about 4 seconds to about 12 seconds, about 5 seconds to about 6 seconds, about 5 seconds to about 7 seconds, about 5 seconds to about 8 seconds, about 5 seconds to about 9 seconds, about 5 seconds to about 10 seconds, about 5 seconds to about 11 seconds, about 5 seconds to about 12 seconds, about 6 seconds to about 7 seconds, about 6 seconds to about 8 seconds, about 6 seconds to about 9 seconds, about 6 seconds to about 10 seconds, about 6 seconds to about 11 seconds, about 6 seconds to about 12 seconds, about 7 seconds to about 8 seconds, about 7 seconds to about 9 seconds, about 7 seconds to about 10 seconds, about 7 seconds to about 11 seconds, about 7 seconds to about 12 seconds, about 8 seconds to about 9 seconds, about 8 seconds to about 10 seconds, about 8 seconds to about 11 seconds, about 8 seconds to about 12 seconds, about 9 seconds to about 10 seconds, about 9 seconds to about 11 seconds, about 9 seconds to about 12 seconds, about 10 seconds to about 11 seconds, about 10 seconds to about 12 seconds, or about 11 seconds to about 12 seconds. In some aspects, the target cooling temperature is maintained for about 1 second, about 2 seconds, about 3 seconds, about 4 seconds, about 5 seconds, about 6 seconds, about 7 seconds, about 8 seconds, about 9 seconds, about 10 seconds, about 11 seconds, or about 12 seconds. In some aspects, the target cooling temperature is maintained for at least about 1 second, about 2 seconds, about 3 seconds, about 4 seconds, about 5 seconds, about 6 seconds, about 7 seconds, about 8 seconds, about 9 seconds, about 10 seconds, or about 11 seconds. In some aspects, the target cooling temperature is maintained for at most about 2 seconds, about 3 seconds, about 4 seconds, about 5 seconds, about 6 seconds, about 7 seconds, about 8 seconds, about 9 seconds, about 10 seconds, about 11 seconds, or about 12 seconds.

In some aspects, the target cooling temperature is maintained for about 10 seconds to about 60 seconds. In some aspects, the target cooling temperature is maintained for about 10 seconds to about 15 seconds, about 10 seconds to about 18 seconds, about 10 seconds to about 20 seconds, about 10 seconds to about 25 seconds, about 10 seconds to about 30 seconds, about 10 seconds to about 35 seconds, about 10 seconds to about 40 seconds, about 10 seconds to about 45 seconds, about 10 seconds to about 50 seconds, about 10 seconds to about 55 seconds, about 10 seconds to about 60 seconds, about 15 seconds to about 18 seconds, about 15 seconds to about 20 seconds, about 15 seconds to about 25 seconds, about 15 seconds to about 30 seconds, about 15 seconds to about 35 seconds, about 15 seconds to about 40 seconds, about 15 seconds to about 45 seconds, about 15 seconds to about 50 seconds, about 15 seconds to about 55 seconds, about 15 seconds to about 60 seconds, about 18 seconds to about 20 seconds, about 18 seconds to about 25 seconds, about 18 seconds to about 30 seconds, about 18 seconds to about 35 seconds, about 18 seconds to about 40 seconds, about 18 seconds to about 45 seconds, about 18 seconds to about 50 seconds, about 18 seconds to about 55 seconds, about 18 seconds to about 60 seconds, about 20 seconds to about 25 seconds, about 20 seconds to about 30 seconds, about 20 seconds to about 35 seconds, about 20 seconds to about 40 seconds, about 20 seconds to about 45 seconds, about 20 seconds to about 50 seconds, about 20 seconds to about 55 seconds, about 20 seconds to about 60 seconds, about 25 seconds to about 30 seconds, about 25 seconds to about 35 seconds, about 25 seconds to about 40 seconds, about 25 seconds to about 45 seconds, about 25 seconds to about 50 seconds, about 25 seconds to about 55 seconds, about 25 seconds to about 60 seconds, about 30 seconds to about 35 seconds, about 30 seconds to about 40 seconds, about 30 seconds to about 45 seconds, about 30 seconds to about 50 seconds, about 30 seconds to about 55 seconds, about 30 seconds to about 60 seconds, about 35 seconds to about 40 seconds, about 35 seconds to about 45 seconds, about 35 seconds to about 50 seconds, about 35 seconds to about 55 seconds, about 35 seconds to about 60 seconds, about 40 seconds to about 45 seconds, about 40 seconds to about 50 seconds, about 40 seconds to about 55 seconds, about 40 seconds to about 60 seconds, about 45 seconds to about 50 seconds, about 45 seconds to about 55 seconds, about 45 seconds to about 60 seconds, about 50 seconds to about 55 seconds, about 50 seconds to about 60 seconds, or about 55 seconds to about 60 seconds. In some aspects, the target cooling temperature is maintained for about 10 seconds, about 15 seconds, about 18 seconds, about 20 seconds, about 25 seconds, about 30 seconds, about 35 seconds, about 40 seconds, about 45 seconds, about 50 seconds, about 55 seconds, or about 60 seconds. In some aspects, the target cooling temperature is maintained for at least about 10 seconds, about 15 seconds, about 18 seconds, about 20 seconds, about 25 seconds, about 30 seconds, about 35 seconds, about 40 seconds, about 45 seconds, about 50 seconds, or about 55 seconds. In some aspects, the target cooling temperature is maintained for at most about 15 seconds, about 18 seconds, about 20 seconds, about 25 seconds, about 30 seconds, about 35 seconds, about 40 seconds, about 45 seconds, about 50 seconds, about 55 seconds, or about 60 seconds.

In some aspects, a controlled cooling device is applied over multiple cooling cycles (e.g. the temperature is held to 3° C. for 5 seconds and the target area is allowed to warm for 3 seconds before a subsequent cycle of 3° C. for 5 seconds). In some aspects, the target temperature and cooling duration can be independently selected for each cooling cycle. In some aspects, the cooling device can be applied over 1, 2, 5, 10, 20, 50, or 100 cooling cycles. In some aspects, the time between cycles can be about 1, about 2, about 3, about 4, about 5, about 10, about 30, or about 60 seconds. In some aspects, cooling cycles may be described as a duty cycle (ratio of cooling time to warming time) of about 10%, about 20%, about 30%, about 40%, about 50%, about 60%, about 70%, about 80%, about 90%, or about 100%.

In some aspects, the angle of inclination and the orientation are selected to minimize a reduction of cryogen flow or maximize the cryogen flow into the cooling device caused by gravity. In some instances, the angle of inclination and the orientation are selected to optimize discharge pressure of the cryogen in the handheld cooling device. In some instances, the angle of inclination and the orientation are selected to optimize discharged amount of the cryogen to the target surface per application.

In some aspects, a treatment to the target surface can be concurrently or near simultaneously (e.g., within about 1 second, about 2 seconds, about 3 seconds, about 4 seconds, about 5 seconds, about 10 seconds after cooling the target surface or before cooling the target surface) applied with the cooling of the target surface. In some aspects, the treatment is performed through the opened portion of the inclined applicator tip. In some aspects, the treatment comprises an injection, a laser treatment, an energy based device treatment, a surgery, or a drug application. For example, a cooling treatment can be combined with an injection in order to provide a vaccination to a subject with minimal pain or swelling at the injection site. A single technician or a team of two or more technicians can administer the vaccine by first applying the cooling device by placing the applicator tip onto a target surface of the subject. The technician or a second technician can then inject either during cooling by the cooling device or by injecting within a few second of the conclusion of a cooling cycle.

In some aspects, cooling treatment can be applied alone using a controlled cooling device to reduce swelling and/or inflammation, to reduce bruising or hematoma, to reduce pain at a target site, as an analgesic, to relax a subject, to make the procedure more comfortable, or combinations thereof. In some aspects, cooling treatment can be applied in combination with one or more injections or biopsies using one or more needles.

In some aspects, the injections can comprise intramuscular, subcutaneous, intradermal, intraperitoneal, intraosseous, intracardiac, intraarticular, or extracavernous injections, and combinations thereof.

In some aspects, the injections can comprise a vaccination, a vitamin injection, a therapeutic agent injection, a Botox injection, or injections of Methionine, Inositol, Choline, magnesium, glutathione, zinc, iron, ozone, nicotinamide, NAD+, NADH, Pepcid, Zofran, or IV fluids (e.g. for hydration), and combinations of two or more thereof.

In some aspects, vitamin injections can comprise A vitamins, B Vitamins, Vitamin C (ascorbic acid), choline, Vitamin D (calciferol), Vitamin E (alpha-tocopherol), Vitamin K (phylloquinone, menadione), B1 (thiamine), B2 (riboflavin), B3 (niacin), B5 (pantothenic acid), B6, B7 (biotin), B12, folic acid, and/or combinations of two or more thereof.

In some aspects, the vaccinations can comprise vaccination against COVID, influenza, measles, varicella, dengue, diphtheria, hepatitis A, hepatitis B, Haemophilus influenzae type b, HPV, meningococcal, mumps, pneumococcal pneumonia, poliomyelitis, rotavirus, rubella, shingles, tetanus, pertussis, adenovirus, anthrax, cholera, Japanese Encephalitis, rabies, smallpox, tuberculosis, typhoid fever, or yellow fever, and combinations of two or more thereof.

In some aspects, the temperature and the cooling duration can vary or optimized based on the size of the injection needle size. For example, the needle gauge can be selected to be any needle gauge suitable for the particular injection/biopsy application (e.g., 30 gauge for Botox injections, 23-25 gauge for subcutaneous vaccinations, 22-25 gauge for intramuscular vaccinations, 14-18 gauge for biopsies, etc.) In some examples, the needle gauge is selected from the group of 34 gauge, 33 gauge, 32 gauge, 31 gauge, 30 gauge, 29 gauge, 28 gauge, 27 gauge, 26s gauge, 26 gauge, 25s gauge, 24 gauge, 23s gauge, 23 gauge, 22s gauge, 22 gauge, 21 gauge, 20 gauge, 19 gauge, 18 gauge, 17 gauge, 16 gauge, 14 gauge, 13 gauge, 12 gauge, 11 gauge, and 10 gauge. Thus, in some examples, the cooling temperature can be set lower and/or the cooling duration can be set longer if the needle size is larger (e.g., lower gauge of needles). For example, if the needle is 25 gauge in size, the cooling temperature can be set at 3° C. and the cooling duration can be set for 5 seconds. For another example, if the needle is 15 gauge in size, the cooling temperature can be set at 3° C. and the cooling duration can be set for 10, 15, 20, 25, or 30 seconds. Alternatively and/or additionally, if the needle is 15 gauge in size, the cooling temperature can be set at 2° C. and the cooling duration can be set for 5, 10, 15, 20, or 25 seconds.

### Inclined Applicator Tip

In one aspect, described herein are inclined applicator tips for use with a cooling device. In some aspects, the cooling device is a handheld cooling device. In some aspects, the cooling device is a therapeutic cooling device. In some aspects, the cooling device can be an electrothermal cooling device. In some aspects, the cooling device can be a cryogenic cooling device. In some aspects, the cooling device can be a non-contact cooling device. In some aspects, the cooling device can be a contact-based cooling device. In some aspects, the cooling device sprays controlled cryogen. In some aspects, the cooling device is any suitable device that can be used with the inclined applicator tip, for example, as described in U.S. Patent Application Pub. Nos. US20190175394A1, US20200309436A1, US20200054483A1, US20210290430A1, US20190167330A1, International Patent Application Pub. Nos. WO2018221848A1, WO2022015066A1, WO2019107607A1, WO2021246580A1, WO2022015067A1, WO2020022858A1, Korean Patent Application NOs. KR20190114710A, KR20190124971A, KR20210101187A, KR20190063724A, KR20190063723A, KR20210030315A, KR20200070200A, KR20190124974A, KR20200012677A, KR20190063722A, KR20190124970A, KR20200124635A, KR20190124973A, KR20190124969A and U.S. Pat. Nos. 11,207,488, 11,154,417, 11,241,332, which are incorporated by references herein.

### Use of Inclined Applicator Tip

In some aspects, the inclined applicator tip provides improved access to the cooling area while using a therapeutic cooling device. In some aspects, the improved access allows for easier combination of cooling treatment (e.g., cooling therapy, cooling anesthesia, analgesic cooling, etc.) with other types of treatment or therapy. In some aspects, the other types of treatment or therapy can be an injection, a laser therapy, an energy-based device therapy. a light-exposure therapy, radiotherapy, biopsy, surgery, or a combination thereof. In some aspects, use of the cooling therapy device in combination with the other type of therapy reduces pain associated with the other type of therapy. In some aspects, use of the cooling therapy device in combination with the other type of therapy increases the efficacy associated with the other type of therapy. In some aspects, co-administration of cooling therapy and one or more other types of therapy are applied simultaneously, or near simultaneously. In some aspects, the target area where the cooling therapy or co-administered therapies are to be applied is pre-marked, such as using a dye, a pen, tape, or other means of identifying a specific target site.

In some aspects, wherein the therapeutic cooling device is a cryogenic cooling device, the angle of inclination is set to an optimum angle such that access is maximized (at larger angles), while loss of cryogen flow due to gravity is minimized (at angles which keep the cooling axis perpendicular to a horizontal target site, or parallel to a vertical target site). In some aspects, the optimum angle of inclination is no more than about 60 degrees for a vertical target site. In some aspects, the optimum angle of inclination depends on the orientation of the target site with respect to the gravitational force vector acting on the cryogen. In some aspects, the angle of inclination is chosen such that the angle between the longest dimension of a cryogen canister and the axis gravitational force vector is no more than about 60 degrees and the canister is oriented such that the outlet port is lower in elevation than the opposite end of the canister.

In some aspects, described herein are methods of cooling a target surface using a handheld cooling device equipped with an inclined applicator tip. In some aspects, the method can include providing an inclined applicator tip that is adapted to be operably coupled to the handheld cooling device. The method can comprise selecting an orientation of the opened portion with respect to a cooling axis of the handheld cooling device, wherein the orientation of the opened portion forms an angle of inclination (θ) with respect to the cooling axis. The method can comprise using the cooling device to cool a target surface by delivering the cooling medium to the target surface along the cooling axis.

In some aspects, selection is performed by mounting the applicator tip to the handheld cooling device in a position selected from three discreet rotational orientations. In some aspects, the selection is performed by selecting an orientation from a continuum of rotational orientations using a rotatable mount of the handheld cooling device.

In some aspects, the cooling device is a cryogenic cooling device wherein the cooling medium is a cryogen, which is selected from liquid helium, liquid nitrogen, liquid argon, liquid CO₂, or liquid O₂.

The method can include selecting an orientation of the cryogenic cooling device to minimize a reduction of cryogen flow into the cooling device caused by gravity. The method can further comprise applying a treatment to the target surface concurrently or near simultaneously with the cooling. The method can include treating a subject by applying cooling and an injection, a laser treatment, an energy based device, a surgery, or a drug application. The method can include treatment that is performed through the opened portion of the inclined applicator tip.

### Methods of Cooling the Target Surface in Combination with Treatment

### Use of a Cryogenic Therapy Device Before and or During a Treatment

A medical professional wants to co-administer cooling therapy and an injection using a cryogenic cooling device. A cryogenic cooling device coupled to or equipped with a non-inclined applicator tip and a cryogen canister is used to apply a cooling jet to a target site along a cooling axis. The enclosed non-inclined applicator tip prevents liquid cryogen from entering the nozzle by gravity (by making the angle too steep) and blocks access to the target site for subsequent injections. In order to use the device, the target site must be oriented close to horizontal, and the cryogenic cooling device must be removed prior to administration of an injection or other subsequent therapy.

When the medical professional uses a cryogenic cooling device equipped with an inclined applicator tip, they can rotate the angle of inclination about the cooling axis of the device such that the cryogenic cooling device will operate on target tissues at any angle. The angle of inclination of the applicator tip is chosen to be as large as possible to maximize access (by placing the cooling farther out of the way of the target site) such that liquid cryogen is not prevented from entering the nozzle by gravity.

The temperature of a target surface is controlled by forming a cold jet of cryogen which is applied using an inclined applicator tip to produce a temperature cold enough to immediately (e.g., within 1 sec, within 2 sec, within 3 sec, within 5 sec, etc.) anesthetize a target area on a subject or provide a reduction in pain, bruising, swelling, and or inflammation of the target area, while allowing access to the target site. Reduction of the temperature mitigates pain in the target site during hypodermic injection and/or laser treatment procedures or energy based device treatment procedure, which are carried out while the cold jet is present, or a few seconds after, for example within 3 to 5 seconds. The inclined applicator is designed to set a constant distance between the cryogen nozzle outlet to promote precision temperature control. The inclined applicator tip has an opened contact surface mounted at an angle to allow maximum visibility and accessibility to the target area while cooling is taking place and/or shortly thereafter.

The medical professional can adjust the cooling temperature and/or duration based on various conditions (e.g., types of treatment, size or types of the injection tools (e.g., needles), age or health condition of the individual, duration of the treatment, etc.). For example, where the injection needle is 25 gauge in size, the medical professional can set or tune the device to cool the target surface to 3° C. for 5 seconds or 1° C. for 4 seconds.

Depending on the number of skin areas to be treated or the number of injections to be performed, the medical professional can choose to either treat each skin area individually (e.g., cool the area with controlled cryogen and perform the injection or treatment at that spot and have the spray stop after that specific skin area is treated) or choose to treat a plurality of skin areas while having the cooling device cool continuously (e.g., cool the first area with controlled cryogen and, while the device is still spraying, move the spray to a second area while performing the injection or treatment on that first area), having the spray turn off after the final skin area to be treated is completed.

Accordingly, different angle of inclinations are chosen depending on whether the target area is vertical (such as target skin for injecting a vaccination into an arm of a standing person) or horizontal. The angle of inclination of a fixed inclined applicator tip can be adjusted with respect to a target site by rotating a rotatable mount on the device about the cooling axis in order to achieve an appropriate angle for the specific target site. Simultaneous injection and simultaneous laser therapy or energy based device therapy while applying a cryotherapy device to reduce pain at the injection or laser application site or energy based device application site can be achieved by methods of treatment described herein. The open shaped ring of the inclined applicator tip maximizes the visibility and accessibility, particularly compared to concentric closed ring applicator tips, which place the device perpendicular to the target site.

In other scenarios, the medical professional can configure the cooling device so that the cools the target skin area to a precise temperature without creating residual ice flakes on the target skin area. These are typically in warmer temperatures (e.g., 2° C., 3° C., 4° C., 5° C., 6° C., 7° C., 8° C., 9° C., 10° C.). In such scenarios, these temperatures ranges offer an improved reduction in pain alongside certain treatments while maximizing the medical professional's ability to see the target area to ensure that the appropriate area is treated or that the treatment is not interfered with (e.g., lasering the appropriate area, applying the RF microneedling to the right area, injecting the right area, etc.). For example, the medical professional doing a lasering treatment can configure a cooling device so that a sprayed controlled cryogen cools the target area to 4° C. so that an energy device that directs heating energy to the skin is not interfered by minimal or no formation of ice flakes on the target area. In some embodiments, the medical professional can adjust the cooling device so that the target area is cooled to different temperatures for different times. By way of example, the medical professional can configure the cooling device so that the sprayed controlled cryogen first cools the target area to a first temperature (e.g., −2° C., −1° C., 0, 1° C.) and maintain that temperature for a first period of time (e.g., 1 second, 2 seconds, 3 seconds, 4 seconds, 5 seconds, or warmer) and then automatically transitions to cool the target area to a second temperature that is warmer (e.g., 4° C., 5° C., 6° C., 7° C. 8° C. 9° C., 10° C.,) for a second period of time (e.g., 1 second, 2 seconds, 3 seconds, 4 seconds, 5 seconds, or longer). In such a scenario, the first, colder temperature allows for a maximizing of reduction in pain or vasoconstriction (to help reduce bruising), while the second, warmer temperature allows for a minimizing of ice flake formation so the medical professional can more easily see the area to be treated and perform the procedure. The medical professional can adjust the first and second temperature and first and second times depending on the nature of their procedure, patient preference (e.g., they like colder or warmer sensations) or their preferred workflow (e.g., the professional prioritizes speed).

### Use of a Cryogenic Therapy Device after Application of a Treatment

A medical professional seeks to reduce swelling and/or inflammation, or seeks to increase patient comfort or to relax a subject after the subject has been treated by another medical procedure (e.g. Botox treatment, laser therapy, an energy based device treatment, vaccination, injection of a therapeutic agent, etc.). The medical professional uses cryogenic cooling device coupled to or equipped with a non-inclined applicator tip and a cryogen canister is used to apply a cooling jet to a target site along a cooling axis. The enclosed non-inclined applicator tip prevents liquid cryogen from entering the nozzle by gravity (by making the angle too steep) and blocks access to the target site for subsequent injections. In order to use the device, the target site must be oriented close to horizontal.

When the medical professional uses a cryogenic cooling device equipped with an inclined applicator tip, they can rotate the angle of inclination about the cooling axis of the device such that the cryogenic cooling device will operate on target tissues at any angle. The angle of inclination of the applicator tip is chosen to be as large as possible to maximize access (by placing the cooling farther out of the way of the target site) such that liquid cryogen is not prevented from entering the nozzle by gravity.

The temperature of a target surface is controlled by forming a cold jet of cryogen which is applied using an inclined applicator tip to produce a temperature cold enough to immediately (e.g., within 1 sec, within 2 sec, within 3 sec, within 5 sec, etc.) anesthetize a target area on a subject, while reducing swelling and/or inflammation, and provides a soothing sensation to the subject. Reduction of the temperature mitigates pain in the target site.

The medical professional can adjust the cooling temperature and/or duration based on various conditions (e.g., type of the prior treatment, size or types of the injection tools used in the prior treatment (e.g., needles), age or health condition of the individual, duration of the treatment, etc.). For example, where the injection needle is 25 gauge in size, the medical professional can set the device to cool the target surface to 5° C. for 3 seconds for post-treatment cooling.

### CLINICAL EXAMPLES

### Clinical Example 1: Protocols for Neuromodulator Injections

### Clinical Example 1A: Simultaneous Cooling & Injection (Recommended; May Require Assistant)

A medical professional provides neuromodulator injections to a subject in need thereof, with the help of an assistant by the following protocol:
1. The medical professional seats the subject in an upright position.
2. The medical professional cleans the treatment area and marks intended injection points with a washable skin marker.
3. The medical professional prepares neuromodulator injections in standard fashion for treating the patient.
4. The medical professional sets a target temperature of a cooling device to 3° C. and a cooling time of the cooling device to 2 seconds at target temperature. The medical professional is free to adjust the temperature and time depending on the patient (e.g., if the patient likes the sensation to be colder, the temperature can be set to 1° C.; if the patient still feels uncomfortable from the poke from previous treatments, the time can be set to 4 seconds to allow for the area to be cooled for longer; if the patient is a darker skin type that is more likely to experience post-inflammatory hyperpigmentation or other skin issues that may arise, the provider can alter the temperature to a warmer temperature like 4° C.).
5. The medical professional reclines the subject back approximately 30 degrees.
6. The medical professional demonstrates the cooling device on a dorsal forearm of the subject so that they are aware of the sound and sensation of the device prior to use of the cooling device on the treatment area. The medical professional may spray first the air before spraying on the subject skin or first spray the professional's own palm before spraying on the patient's skin to mitigate the chance of the subject from being startled by a sudden jet sound.
7. An assistant holds the cooling device lightly to the skin surface, making sure there is full contact with the entire tip. The assistant aligns the marked injection point at the center of the open end of an applicator tip of the cooling device, ensuring that a cryogen cartridge of the cooling device is pointed downwards at a 45-90 degree angle with respect to gravity.
8. The assistant starts the cooling spray by pressing a trigger of the cooling device for 0.5 seconds, which starts a cryogen spray which continues until the trigger is pressed again for 0.5 seconds to stop the spray.
9. When the device reaches the target temperature, a first beep is emitted. The assistant allows the spray to continue for an additional 2 seconds, after the first beep (which is indicated to the assistant by a second, lower-pitched beep emitted from the cooling device).
10. Once the second beep is heard, the medical professional immediately inserts an injection needle loaded with the neuromodulator into the skin of the subject through the marked injection point, delivering the neurotoxin to the treatment area. A cryogen spray may continue even after the second beep for approximately 1, 2, 3, or 4 seconds, during which the medical professional may insert an injection needle.
11. If additional injection points remain, the assistant then moves the cooling device to the next point while the device is still spraying, and steps 7-10 are repeated. Once all treatment points have been injected, the assistant presses the trigger of the cooling device again to stop the spray.
12. The assistant reconfigures the cooling device for post-treatment cooling to minimize inflammation at the treatment area and to minimize bruising. The assistant sets the cooling device to a target temperature of 5° C. and 3 seconds at the target temperature.
13. Once all treatment sites have been cooled, the assistant applies an additional post-treatment cooling spray to the entire region to relax the subject and enhance their treatment experience using a 5° C. target temperature with 2 seconds at the target temperature.

### Clinical Example 1B: Simultaneous Cooling & Injection (Recommended; May Require Assistant)

A medical professional provides neuromodulator injections to a subject in need thereof, by the following protocol:

### Non-simultaneous Cooling & Injection (Recommended for Providers without Assistance)

1. The medical professional seats the subject in an upright position.
2. The medical professional cleans the treatment area and marks intended injection points with a washable skin marker.
3. The medical professional prepares neuromodulator injections in standard fashion for treating the patient.
4. The medical professional sets a target temperature of a cooling device to 1° C. and a cooling time of the cooling device to 5 seconds at target temperature (or 3° C. for 5 seconds if treating certain areas of the skin, such as the hairline, if the patient prefers).
5. The medical professional reclines the subject back approximately 30 degrees.
6. The medical professional demonstrates the cooling device on a dorsal forearm of the subject so that they are aware of the sound and sensation of the device prior to use of the cooling device on the treatment area.
7. The medical professional holds the cooling device lightly to the skin surface, making sure there is full contact with the entire tip. The assistant aligns the marked injection point at the center of the open end of an applicator tip of the cooling device, ensuring that a cryogen cartridge of the cooling device is pointed downwards at a 45-90 degree angle with respect to gravity.
8. The medical professional starts the cooling spray by pressing a trigger of the cooling device for 0.5 seconds, which starts a cryogen spray which continues until the trigger is pressed again for 0.5 seconds to stop the spray.
9. When the device reaches the target temperature, a first beep is emitted. The assistant allows the spray to continue for an additional 5 seconds, after the first beep (which is indicated to the assistant by a second, lower-pitched beep emitted from the cooling device).
10. Once the second beep is heard, the medical professional immediately inserts an injection needle loaded with the neuromodulator into the skin of the subject through the marked injection point, delivering the neurotoxin to the treatment area. A cryogen spray may continue even after the second beep for approximately 1 second, during which the medical professional may insert an injection needle. In this instance, the device stops the spray of the controlled cryogen automatically.
11. If additional injection points remain, the medical professional repeats steps 7-10 for each treatment point.
12. The medical professional then reconfigures the cooling device for post-treatment cooling to minimize inflammation at the treatment area and to minimize bruising. The medical professional sets the cooling device to a target temperature of 5° C. and 3 seconds at the target temperature.
13. Once all treatment sites have been cooled, the assistant applies an additional post-treatment cooling spray to the entire region to relax the subject and enhance their treatment experience using a 5° C. target temperature with 2 seconds at the target temperature.

### Clinical Example 2: Protocols for Dermal Filler Injections

### Dermal Filler Injections

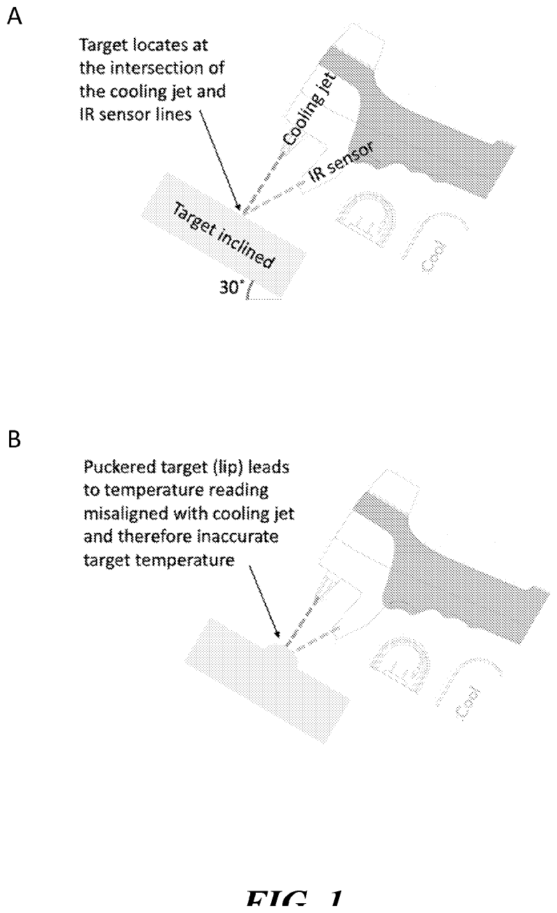

1. The medical professional seats the subject in an upright position.
2. The medical professional cleans the treatment area and, if appropriate, marks intended injection points with a washable skin marker.
3. The medical professional sets a target temperature of a cooling device to 3° C. and a cooling time of the cooling device to 2 seconds at target temperature.
4. The medical professional reclines the subject back approximately 30 degrees.
5. The medical professional demonstrates the cooling device on a dorsal forearm of the subject so that they are aware of the sound and sensation of the device prior to use of the cooling device on the treatment area.
6. The medical professional indicates to an assistant where they will be injecting by either specifying a previously marked injection point, or by pointing with an injection needle tip.
7. The assistant holds the cooling device lightly to the skin surface, making sure there is full contact with the tip. The assistant aligns the marked injection point at the center of the open end of an applicator tip of the cooling device, ensuring that a cryogen cartridge of the cooling device is pointed downwards at a 45-90 degree angle with respect to gravity. The assistant ensures the tip is flush with the skin, but does not apply excess pressure to the skin so that it remains flat FIG. 1A. Excess pressure creates puckering or other distortion of the skin at the injection site which can misalign the temperature sensor from the intended cooling point as shown in FIG. 1B. In other situations, the assistant may hold the cooling device so that no part of the device physically comes into contact with the skin but the tip is hovering just over the skin area (e.g., 2 or 3 mm). This technique of hovering or floating may be helpful if the assistant knows that they will need to quickly move to a new area and wants to avoid touching the skin surface to speed up the process and to also avoid distorting the original contour of the skin (so the assistant may be understand how much filler to inject in later steps).
8. The assistant starts the cooling spray by pressing a trigger of the cooling device for 0.5 seconds, which starts a cryogen spray which continues until the trigger is pressed again for 0.5 seconds to stop the spray.
9. When the device reaches the target temperature, a first beep is emitted. The assistant allows the spray to continue for an additional 2 seconds, after the first beep (which is indicated to the assistant by a second, lower-pitched beep emitted from the cooling device).
10. Once the second beep is heard, the medical professional immediately inserts an injection needle into the skin of the subject through the marked injection point, delivering the dermal filler to the treatment area.
11. If additional injection points remain, the assistant then moves the cooling device to the next point, and steps 7-10 are repeated. Once all treatment points have been injected, the assistant presses the trigger of the cooling device again to stop the spray.
12. The assistant reconfigures the cooling device for post-treatment cooling to minimize inflammation at the treatment area and to minimize bruising. The assistant sets the cooling device to a target temperature of 5° C. and 3 seconds at the target temperature.
13. Once all treatment sites have been cooled, the assistant applies an additional post-treatment cooling spray to the entire region to relax the subject and enhance their treatment experience using a 5° C. target temperature with 2 seconds at the target temperature.

### Clinical Example 3: Platelet Rich Plasma Scalp Injections

1. A medical professional draws blood and prepares platelet rich plasma for injection in standard fashion
2. The medical professional seats the subject in an upright position.
3. The medical professional cleans the treatment area and, if appropriate, marks intended injection points with a washable skin marker.
4. The medical professional sets a target temperature of a cooling device to 3° C. and a cooling time of the cooling device to 2 seconds at target temperature.
5. The medical professional reclines the subject back approximately 30 degrees.
6. The medical professional demonstrates the cooling device on a dorsal forearm of the subject so that they are aware of the sound and sensation of the device prior to use of the cooling device on the treatment area.
7. The medical professional holds the cooling device with their non-dominant hand, placing it on the scalp wherein the scalp is in full contact with an edge of the tip and the cryogenic cartridge is pointed downwards at a 45-90 degree angle with respect to gravity. The medical professional moves hairs away from the cooling path as much as possible to prevent interference with direct cooling of the scalp. In other instances, the medical professional will utilize the inclined applicator tip to part hair and fix hair in a specific location to facilitate access to the scalp during the cooling spray to follow. In other instances, the medical professional may work with one or more assistants—one responsible for parting hair, another responsible for handling and orienting the device, and another one responsible for performing the PRP injection into the scalp.
8. The medical professional starts the cooling spray by pressing a trigger of the cooling device for 0.5 seconds, which starts a cryogen spray which continues until the trigger is pressed again for 0.5 seconds to stop the spray.
9. When the device reaches the target temperature, a first beep is emitted. The assistant allows the spray to continue for an additional 5 seconds, after the first beep (which is indicated to the assistant by a second, lower-pitched beep emitted from the cooling device).
10. Once the second beep is heard, the medical professional presses the trigger again to stop the cooling spray, removes the device and immediately inserts an injection needle loaded platelet rich plasma into the scalp of the subject, injecting platelet rich plasma into the subcutaneous tissue. A cryogen spray may continue even after the second beep for approximately 1, 2, 3, or 4 seconds, during which the medical professional may insert an injection needle.
11. If additional injection points remain, the medical professional repeats steps 7-10 for each treatment point.
12. The medical professional then reconfigures the cooling device for post-treatment cooling to minimize inflammation at the treatment area and to minimize bruising. The medical professional sets the cooling device to a target temperature of −1° C. and 3 seconds at the target temperature.
13. The medical professional then applies a post-treatment cooling spray to the entire scalp to minimize bruising and prevent swelling.

### Clinical Example 4: Vaccinations

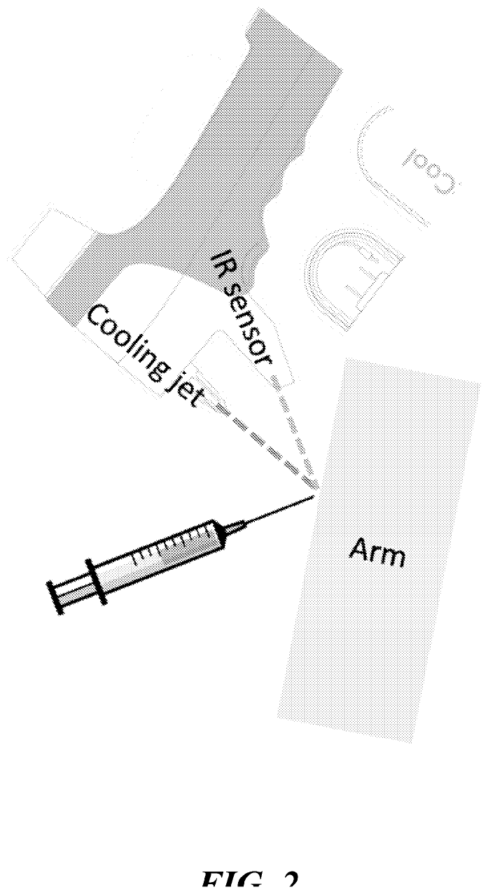

1. A medical professional sets the target temperature of a cooling device to 3° C. with a cooling time of 5 seconds at the target temperature.
2. The medical professional positions a subject in preparation for vaccination and cleans the intended injection point.
3. With their non-dominant hand, the medical professional holds the cooling device lightly to the skin surface of the subject, making sure there is full contact with the entire applicator tip and that the cryogen cartridge of the cooling device is angled downwards as shown in FIG. 2.
4. The medical professional starts the cooling spray by pressing a trigger of the cooling device for 0.5 seconds, which starts a cryogen spray which continues until the trigger is pressed again for 0.5 seconds to stop the spray.
5. When the device reaches the target temperature, a first beep is emitted. The assistant allows the spray to continue for an additional 5 seconds, after the first beep (which is indicated to the assistant by a second, lower-pitched beep emitted from the cooling device).
6. Once the second beep is heard, the medical professional immediately inserts an injection needle loaded with the vaccine into the skin of the subject through the marked injection point, delivering the vaccination. A cryogen spray may continue even after the second beep for approximately 1, 2, 3, or 4 seconds, during which the medical professional may insert an injection needle.
7. The medical professional then presses the trigger again to stop the cooling spray and removes the device.

### Clinical Example 5: Freezing Mode (for the Destruction of Actinic Keratoses, Verrucae, and Benign Skin Lesions)

8. A medical professional sets a cooling device to Freezing Mode and assess a lesion of interest, noting its diameter and approximate depth. The medical professional then consults a reference chart to determine the appropriate freezing time based on the lesion characteristics. The medical professional then configures the cooling device accordingly, and treats the lesion by application of the cooling device. A medical professional may refer the time duration of each freezing period and of total freezing duration on the device LCD screen.

### Clinical Example 6: Protocol for Skin Brightening, the “Cool Facial”

1. A technician cleanses the face of a subject to remove all topical agents (ex. Make-up, moisturizer, sunscreen) and thoroughly dries the treatment area.
2. The technician reclines the subject backwards until they are resting comfortably flat.
3. The technician configures a cooling device to a 0° C. target temperature with 3 seconds at the target temperature.
4. The technician demonstrates the device on the subject's dorsal forearm so that they are aware of the sound and sensation of the operation of the device.
5. The technician holds the device lightly to the skin surface, making sure there is full contact with the entire applicator tip of the device and that the cryogen cartridge of the device is pointed downwards at a 45-90 degree angle with respect to gravity.
6. The technician starts the cooling spray by firmly pressing the trigger for 0.5 seconds.
7. Once the first beep is heard, the target temperature is reached. The technician continues the spray for an additional 3 seconds, which is indicated by a second, lower-pitched beep.
8. Once the second beep is heard, the technician glides the device to the adjacent skin to begin cooling the next area, which is the next non-overlapping, cooling spray treatment point.
9. The technician repeats steps 5-8 until the entire facial region or part of facial region has been treated, while taking care not to treat mucosal surfaces.

### Clinical Example 7: Vitamin B12 Injections

1. A medical professional sets the target temperature of a cooling device to 3° C. with a cooling time of 5 seconds at the target temperature.
2. The medical professional positions a subject in preparation for vaccination and cleans the intended injection point.
3. With their non-dominant hand, the medical professional holds the cooling device lightly to the skin surface of the subject, making sure there is full contact with the entire applicator tip and that the cryogen cartridge of the cooling device is angled downwards as shown in FIG. 2. In some embodiments, the medical professional orients the device on the patient's deltoid and orients the applicator tip so that the opened portion is facing downward to allow easier access for this medical professional to access the injection site coming from the bottom.
4. The medical professional starts the cooling spray by pressing a trigger of the cooling device for 0.5 seconds, which starts a cryogen spray which continues until the trigger is pressed again for 0.5 seconds to stop the spray.
5. When the device reaches the target temperature, a first beep is emitted. The assistant allows the spray to continue for an additional 5 seconds, after the first beep (which is indicated to the assistant by a second, lower-pitched beep emitted from the cooling device).
6. Once the second beep is heard, the medical professional immediately inserts an injection needle loaded with the vaccine into the skin of the subject through the marked injection point, delivering the vaccination. A cryogen spray may continue even after the second beep for approximately 1, 2, 3, or 4 seconds, during which the medical professional may insert an injection needle.
7. The medical professional then presses the trigger again to stop the cooling spray and removes the device.

### Clinical Example 8: Blood Draw Injections

1. A medical professional prepares for the blood draw in standard fashion.
2. The medical professional seats the subject in an upright position.
3. The medical professional cleans the treatment area (near the antecubital fossa vein near the elbow) and, if appropriate, marks intended injection points with a washable skin marker.
4. The medical professional sets a target temperature of a cooling device to 3° C. and a cooling time of the cooling device to 5 seconds at target temperature. The medical professional selects 3° C. as a starting temperature because the subject's skin type is IV and understands to use a slightly warmer temperature to mitigate concerns of post-inflammatory hyperpigmentation.
5. The medical professional demonstrates the cooling device on a dorsal forearm of the subject so that they are aware of the sound and sensation of the device prior to use of the cooling device on the treatment area.
6. The medical professional holds the cooling device with their non-dominant hand, placing it over the vein. While the professional ensures that the device is in full contact with the skin and that the cryogenic cartridge is pointed downwards at a 45-90 degree angle with respect to gravity, the professional also ensures that the device is resting gently to mitigate any puckering.
7. The medical professional starts the cooling spray by pressing a trigger of the cooling device for 0.5 seconds, which starts a controlled cryogen spray which continues until the trigger is pressed again for 0.5 seconds to stop the spray.
8. When the device reaches the target temperature, a first beep is emitted. The assistant allows the spray to continue for an additional 5 seconds, after the first beep (which is indicated to the assistant by a second, lower-pitched beep emitted from the cooling device).
9. Once the second beep is heard, the medical professional immediately inserts an injection needle into the vein of the subject. A cryogen spray may continue even after the second beep for approximately 1, 2, 3, or 4 seconds, during which the medical professional may insert an injection needle.

While preferred aspects of the present invention have been shown and described herein, it will be obvious to those skilled in the art that such aspects are provided by way of example only. It is not intended that the invention be limited by the specific examples provided within the specification. While the invention has been described with reference to the aforementioned specification, the descriptions and illustrations of the aspects herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the aspects of the invention described herein can be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### Additional Embodiments

1. A method of reducing discomfort of a subject during a treatment to a skin area of the subject, the method comprising:
   a) providing a handheld cooling device, wherein the cooling device comprises an inclined applicator tip, wherein the inclined applicator tip comprises an opened portion, an elongated body portion coupled to the opened portion, and a mounting portion adapted to be operably coupled to the handheld cooling device;
   b) contacting the applicator tip to or around the skin area;
   c) cooling the skin area to a cooling temperature; and
   d) applying the treatment to the skin area by providing an access of the treatment through the opened portion.
2. The method of embodiment 1, wherein the treatment is an injection, a lip filler treatment, a piercing treatment, a laser treatment, an energy based device treatment, or a surgery.
3. The method of embodiment 1 or 2, wherein the skin area comprises irregular surface.
4. The method of any one of embodiments 1-3, wherein the skin area is a lip, eyelid, ear, nose, face, or neck.
5. The method of any one of embodiments 1-4, wherein the contacting comprises contacting a tip of the opened portion to or around the skin area.
6. The method of embodiment 5, wherein the circumference of the opened portion partially surrounds the skin area.
7. The method of embodiment 6, wherein the circumference of the opened portion is within 5 mm, 10 mm, 15 mm, 20 mm, or 25 mm from the skin area of the treatment.
8. The method of any one of embodiments 1-7, wherein the cooling temperature is between about 0 degree to 10 degree Celsius.
9. The method of any one of embodiments 1-8, wherein the cooling temperature is between 3 degree to 8 degree Celsius.
10. The method of any one of embodiments 1-9, wherein the temperature of the skin area is maintained at the cooling temperature for at least 2 sec, 5 sec, 10 sec, 15 sec, or 20 sec.
11. The method of embodiment 1, further comprising selecting an orientation of the opened portion with respect to a cooling axis of the handheld cooling device, wherein the orientation of the opened portion forms an angle of inclination (θ) with respect to the cooling axis.
12. The method of embodiment 8, the angle of inclination and the selected orientation are designed to minimize a reduction of cryogen flow into the handheld cooling device caused by gravity.
13. A method of reducing discomfort of a subject after a treatment to a skin area of the subject, the method comprising:
   a) providing a handheld cooling device, wherein the cooling device comprises an inclined applicator tip, wherein the inclined applicator tip comprises an opened portion, an elongated body portion coupled to the opened portion, and a mounting portion adapted to be operably coupled to the handheld cooling device;
   b) contacting the applicator tip to or around the skin area;
   c) applying the treatment to the skin area by providing an access of the treatment through the opened portion; and
   d) cooling the skin area to a cooling temperature.
14. The method of embodiment 13, wherein the treatment is an injection, a laser treatment, an energy based device treatment, or a surgery.
15. The method of embodiment 13 or 14, wherein the discomfort comprises swelling of the skin area or a pain in the skin area.
16. The method of any one of embodiments 13-15, wherein the skin area comprises irregular surface.
17. The method of any one of embodiments 13-16, wherein the skin area is eyelid, ear, nose, face, or neck.
18. The method of any one of embodiments 13-17, wherein the contacting comprises contacting a tip of the opened portion to or around the skin area.
19. The method of embodiment 18, wherein the circumference of the opened portion partially surrounds the skin area.
20. The method of embodiment 19, wherein the circumference of the opened portion is within 5 mm, 10 mm, 15 mm, 20 mm, or 25 mm from the skin area of the treatment.
21. The method of any one of embodiments 13-20, wherein the cooling temperature is between about 0 degree to 10 degree Celsius.
22. The method of any one of embodiments 13-21, wherein the cooling temperature is between 3 degree to 8 degree Celsius.
23. The method of any one of embodiments 13-22, wherein the temperature of the skin area is maintained at the cooling temperature for at least 1 sec, 2 sec, 5 sec, 10 sec, 15 sec, or 20 sec.
24. The method of any one of embodiments 13-23, wherein the step d) is performed within 1 sec, 2 sec, 3 sec, 4 sec, 5 sec, 10 sec, or 15 sec after the treatment.
25. The method of any one of embodiments 13-24, wherein the step d) is performed for a duration of 1 sec, 2 sec, 3 sec, 4 sec, 5 sec, 10 sec, or 15 sec.
26. The method of any one of embodiments 13-25, wherein step d) is performed plurality of times with an intermittent break period.
27. The method of embodiment 26, wherein the intermittent break period is at least 1 sec, 2 sec, 5 sec, 10 sec, 15 sec, or 20 sec.
28. The method of embodiment 26 or 27, wherein step d) is performed for the same duration for the plurality of times.
29. The method of embodiment 26 or 27, wherein step d) is performed for the different durations.
30. A method of administering an injection to a subject in need thereof, the method comprising:
   a) preparing a treatment area, wherein preparing comprises cleaning the area;
   b) preparing the injection (e.g. loading a syringe);
   c) setting a target temperature and a cooling duration of a handheld cooling device;
   d) holding the handheld cooling device such that an applicator tip of the handheld cooling device is in contact with the treatment area;
   e) activating the handheld cooling device; and
   f) upon indication from the handheld cooling device or after a predetermined period of time, injecting through skin of the injection site;
31. The method of embodiment 30, wherein preparing the treatment area further comprises marking a point of injection with a washable skin marker.
32. The method of embodiment 31, wherein holding the handheld cooling device is performed such that the marked injection point is approximately centered within the applicator tip of the handheld cooling device.
33. The method of any of the preceding embodiments, wherein the target temperature is set to no more than about 5° C. and the cooling duration is set to no more than about 5 seconds.
34. The method of any of the preceding embodiments, wherein the target temperature is set to about 5° C. and the cooling duration is set to about 3 seconds.
35. The method of any of embodiments 30-34, wherein the target temperature is set to about 5° C. and the cooling duration is set to about 2 seconds.
36. The method of any of embodiments 30-34, wherein the target temperature is set to about 3° C. and the cooling duration is set to no more than about 5 seconds.
37. The method of any of embodiments 30-34, wherein the target temperature is set to about 3° C. and the cooling duration is set to about 2 seconds.
38. The method of any of the preceding embodiments, wherein the holding and/or the injection are performed by an assistant.
39. The method of any of the preceding embodiments, wherein the injection is performed during a cooling cycle of the handheld cooling device.
40. The method of any of embodiments 30-39, wherein the injection is performed after the completion of a cooling cycle of the handheld cooling device.
41. The method of any of the preceding embodiments, wherein the subject is a patient seated in an upright position.
42. The method of any of the preceding embodiments, wherein the method further comprises reclining the patient back about 30° from an upright position.
43. The method of any of the preceding embodiments, wherein the indication from the handheld cooling device is an audible beep, an illumination of a light, or a haptic feedback.
44. The method of any of the preceding embodiments, wherein the contact between the applicator tip and the treatment area is such that a contact surface of the applicator tip is in complete contact with a surface of the treatment area, but does not create surface pressure sufficient to cause puckering or other distortion to a shape of the surface.
45. The method of any of the preceding embodiments, wherein the method further comprises, prior to treatment, demonstrating the cooling device to the subject on part of the subject different from the treatment area.
46. The method of any of the preceding embodiments, wherein the injection is a neuromodulator injection, a vaccination, or a platelet rich plasma scalp injection.
47. The method of any of the preceding embodiments, wherein the holding places the handheld cooling device at a 45-90 degree angle with respect to a surface of the treatment area.
48. The method of any of the preceding embodiments, wherein the method further comprises post-injection application of the handheld cooling device, whereby post injection application reduces bruising and/or inflammation of the application area.
49. The method of embodiment 48, wherein the post-injection application uses different settings for target temperature and duration from the pre-injection application.
50. The method of embodiment 48, wherein during the post injection application, the target temperature is set to about-1° C. and the cooling duration is set to about 3 seconds.
51. The method of any of the preceding embodiments, wherein one or more of the method steps are repeated to perform injections at additional treatment areas.
52. A method for freezing lesions in a subject in need thereof, the method comprising:
   providing a handheld cooling device equipped with an applicator tip adapted for freezing a lesion of the subject;
   assessing the lesion of the subject to determine a diameter and approximate depth of the lesion;
   based on the determined diameter and depth of the lesion, configuring the freezing time of the handheld cooling device;
   applying the handheld cooling device to the lesion; and
   activating the handheld cooling device; thereby freezing the lesion.
53. A method for brightening the skin of a subject, the method comprising:
   a) preparing a treatment area, wherein preparing comprises cleansing the skin of the subject to remove topical agents;
   b) setting a target temperature and a cooling duration of a handheld cooling device;
   c) holding the handheld cooling device such that an applicator tip of the handheld cooling device is in contact with a portion of the treatment area;
   d) activating the handheld cooling device; and
   f) repeating steps c) and d) at substantially non-overlapping portions of the treatment area until the entire treatment area is cooled using the handheld cooling device;
54. The method of embodiment 53, wherein the target temperature is set to about 5° C. and the cooling duration is set to about 3 seconds.
55. The method of any of embodiments 53-54, wherein the holding places the handheld cooling device at a 45-90 degree angle with respect to a surface of the treatment area.
56. The method of any of embodiments 53-55, wherein the method further comprises, prior to treatment, demonstrating the cooling device to the subject on part of the subject different from the treatment area.
57. A method of cooling a target surface using a handheld cooling device, comprising:
   providing an inclined applicator tip that is adapted to be operably coupled to the handheld cooling device, wherein the inclined applicator tip comprises an opened portion, an elongated body portion coupled to the opened portion, and a mounting portion adapted to be operably coupled to the handheld cooling device;
   selecting an orientation of the opened portion with respect to a cooling axis of the handheld cooling device, wherein the orientation of the opened portion forms an angle of inclination (θ) with respect to the cooling axis; and
   cooling the target surface by delivering the cooling medium to the target surface along the cooling axis.
58. The method of embodiment 57, wherein selection is performed by mounting the applicator tip to the handheld cooling device in a position selected from three discreet rotational orientations.
59. The method of embodiment 57, wherein the selection is performed by selecting an orientation from a continuum of rotational orientations using a rotatable mount of the handheld cooling device.
60. The method of any one of embodiments 57-59, wherein the cooling medium is a cryogen.
61. The method of embodiments 57-60, wherein the angle of inclination and the user selected orientation are designed to minimize a reduction of cryogen flow into the cooling device caused by gravity.
62. The method of any one of embodiments 57-61, further comprising applying a treatment to the target surface concurrently or near simultaneously with the cooling.
63. The method of embodiment 62, wherein the treatment comprises an injection, a laser treatment, an energy based device treatment, a surgery, or a drug application.
64. The method of any one of embodiments 61-63, wherein the treatment is performed through the opened portion of the inclined applicator tip.

1. A method of rapidly and precisely cooling a skin area of a subject in combination with a treatment of the skin area, the method comprising:
providing a handheld cooling device, wherein the device comprises:
an applicator tip; and
a controlled cryogen spray element configured to spray controlled cryogen; and
contacting the tip on the skin area;
spraying the controlled cryogen onto the skin area; and
applying the treatment to the skin area.

2. The method of claim 1, wherein the treatment comprises an injection, a dermal filler treatment, a neurotoxin treatment, a platelet-rich plasma (PRP) injection, a lidocaine injection, a biopsy, a piercing treatment, a microneedling treatment, a radio frequency (RF) microneedling treatment, a laser treatment, an energy based device treatment, a blood draw, a nerve block, an incision, or a surgery.

3. The method of claim 1, wherein the device further comprises:
a user interface configured to receive a user input of a target temperature;
a real-time temperature sensor configured to detect the real-time temperature of the skin area; and
an alert element configured to provide a target temperature alert in response to the detected real-time temperature of the skin area reaching the target temperature.

4. The method of claim 3, wherein the target temperature alert comprises a visual indicator, an auditory indicator, a physical vibration, a single beep, a plurality of beeps, a visual light, or any combination thereof.

5. The method of claim 3 further comprising receiving the target temperature, detecting the real-time temperature of the skin area, and providing the target temperature alert.

6. The method of claim 3 further comprising applying the treatment after providing the target temperature alert.

7. The method of claim 3, wherein the target temperature comprises −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., or 10° C.

8. The method of claim 3, wherein the user interface is further configured to receive a user input of a duration of time and wherein the alert element is further configured to provide a duration alert in response to the real-time temperature sensor detecting that the skin area has maintained the target temperature for the duration of time.

9. The method of claim 8 further comprising providing the duration alert.

10. The method of claim 9 further comprising applying the treatment after providing the duration alert.

11. The method of claims 8, wherein the duration of time comprises 1 second, 2 seconds, 3 seconds, 4 seconds, 5 seconds, 6 seconds, 7 seconds, 8 seconds, 9 seconds, or 10 seconds.

12. The method of claim 1 further comprising stopping the spray of controlled cryogen onto the skin area before or during the treatment.

13. The method of claim 1, wherein the tip comprises an opened portion.

14. The method of claim 12 further applying the treatment to the skin through the opened portion.

15. The method of claim 1, wherein the device further comprises a cryogen receiving element configured to receive cryogen.

16. The method of claim 15 further orienting the device so the received cryogen flows toward a direction of gravity.

17. The method of claim 1, wherein the skin area comprises a lip, a facial area, a hand, a foot, an arm, a nose, a knee, an elbow, a leg, a body area, a neck area, or a scalp area.

18. The method of claim 1 further comprising replacing the tip that contacted to or around the skin area with a new applicator tip after applying the treatment to the skin area.

19. The method of claim 1, wherein the subject is a human, a mouse, a pig, or a mammal.

20. The method of claim 1 further comprising contacting the tip to a different skin area while the controlled cryogen is spraying, spraying controlled cryogen onto the different skin area, and applying the treatment to the different skin area.
